(19) United States
(12) Patent Application Publication
(10) Pub. No.: US 2005/0143781 A1
(43) Pub. Date: Jun. 30, 2005

(54) Methods and systems for patient adjustment of parameters for an implanted stimulator

(76) Inventors: Rafael Carbunaru, Studio City, CA (US); Jordi Parramon, Valencia, CA (US); Kerry Bradley, Glendale, CA (US)

Correspondence Address: STEVEN L. NICHOLS;RADER, FISHMAN & GRAVER PLLC, 10653 S. RIVER FRONT PARKWAY, SUITE 150, SOUTH JORDAN, UT, 84095, US

(21) Appl. No.: 11/066,962

(22) Filed: Feb. 25, 2005

Related U.S. Application Data
(63) Continuation-in-part of application No. 10/356,414, filed on Jan. 31, 2003.
(63) Continuation-in-part of application No. 10/857,390, filed on May 28, 2004.

(51) Int. Cl. A61N 1/362

(52) U.S. Cl. 607/11; 607/31

(57) ABSTRACT

A method of adjusting operation of a stimulator for treatment of a patient using the stimulator includes adjusting stimulation parameters of the stimulator subsequent to a fitting session with a clinician, where the adjusting is performed by the patient who has the stimulator. A system for patient adjustment of operation of a stimulator used by the patient subsequent to a fitting session with a clinician includes an external device for communicating with the stimulator and a user interface of the external device configured to receive input from the patient to adjust stimulation parameters of the stimulator.

### RELATED APPLICATIONS

The present application is a continuation-in-part, and claims the priority under 35 U.S.C. § 120, of U.S. patent application Ser. No. 10/356,414, filed Jan. 31, 2003, entitled “Patient Programmer for Implantable Devices,” which is incorporated herein by reference in its entirety. The present application is also a continuation-in-part, and claims the priority under 35 U.S.C. § 120, of U.S. patent application Ser. No. 10/857,390, filed May 28, 2004, entitled “Remote Control for Implantable Medical Device,” which is also incorporated herein by reference in its entirety.

### BACKGROUND

Implantable stimulators and microstimulators, also known as BION® devices (where BION® is a registered trademark of Advanced Bionics Corporation, of Valencia, Calif.), are typically characterized by a small, cylindrical housing which contains electronic circuitry that produces electric currents between spaced electrodes. These microstimulators are implanted proximate to target tissue, and the currents produced by the electrodes stimulate the tissue to reduce symptoms or otherwise provide therapy for various disorders. An implantable battery-powered medical device may be used to provide therapy for various purposes including nerve or muscle stimulation. For example, urinary urge incontinence may be treated by stimulating the nerve fibers proximal to the pudendal nerves of the pelvic floor; erectile or other sexual dysfunctions may be treated by providing stimulation of the cavernous nerve(s); and other disorders, e.g., neurological disorders caused by injury or stroke, may be treated by providing stimulation of other appropriate nerve(s).

By way of example, in U.S. Pat. No. 5,312,439, entitled Implantable Device Having an Electrolytic Storage Electrode, an implantable device for tissue stimulation is described. U.S. Pat. No. 5,312,439 is incorporated herein by reference. The described microstimulator shown in the '439 patent relates to an implantable device using one or more exposed, electrolytic electrodes to store electrical energy received by the implanted device, for the purpose of providing electrical energy to at least a portion of the internal electrical circuitry of the implantable device. It uses an electrolytic capacitor electrode to store electrical energy in the electrode when exposed to body fluids.

Another microstimulator known in the art is described in U.S. Pat. No. 5,193,539, “Implantable Microstimulator”, which patent is also incorporated herein by reference. The '539 patent describes a microstimulator in which power and information for operating the microstimulator are received through a modulated, alternating magnetic field in which a coil is adapted to function as the secondary winding of a transformer. The induction coil receives energy from outside the body and a capacitor is used to store electrical energy which is released to the microstimulator's exposed electrodes under the control of electronic control circuitry.

In U.S. Pat. Nos. 5,193,540 and 5,405,367, which patents are incorporated herein by reference, a structure and method of manufacture of an implantable microstimulator is disclosed. The microstimulator has a structure which is manufactured to be substantially encapsulated within a hermetically-sealed housing inert to body fluids, and of a size and shape capable of implantation in a living body, with appropriate surgical tools. Within the microstimulator, an induction coil receives energy from outside the body requiring an external power supply.

In yet another example, U.S. Pat. No. 6,185,452, which patent is likewise incorporated herein by reference, there is disclosed a device configured for implantation beneath a patient's skin for the purpose of nerve or muscle stimulation and/or parameter monitoring and/or data communication. Such a device contains a power source for powering the internal electronic circuitry. Such power supply is a battery that may be externally charged each day. Similar battery specifications are found in U.S. Pat. No. 6,315,721, which patent is additionally incorporated herein by reference.

Other microstimulator systems prevent and/or treat various disorders associated with prolonged inactivity, confinement or immobilization of one or more muscles. Such microstimulators are taught, e.g., in U.S. Pat. No. 6,061,596 (Method for Conditioning Pelvis Musculature Using an Implanted Microstimulator); U.S. Pat. No. 6,051,017 (Implantable Microstimulator and Systems Employing the Same); U.S. Pat. No. 6,175,764 (Implantable Microstimulator System for Producing Repeatable Patterns of Electrical Stimulation; U.S. Pat. No. 6,181,965 (Implantable Microstimulator System for Prevention of Disorders); U.S. Pat. No. 6,185,455 (Methods of Reducing the Incidence of Medical Complications Using Implantable Microstimulators); and U.S. Pat. No. 6,214,032 (System for Implanting a Microstimulator). The applications described in these additional patents, including the power charging techniques, may also be used with the present invention. The '596, '017, '764, '965, '455, and '032 patents are incorporated herein by reference.

### SUMMARY

A method of adjusting operation of a stimulator for treatment of a patient using the stimulator includes adjusting stimulation parameters of the stimulator subsequent to a fitting session with a clinician, where the adjusting is performed by the patient who has the stimulator. A system for patient adjustment of operation of a stimulator used by the patient subsequent to a fitting session with a clinician includes an external device for communicating with the stimulator and a user interface of the external device configured to receive input from the patient to adjust stimulation parameters of the stimulator.

### BRIEF DESCRIPTION OF THE DRAWINGS

The accompanying drawings illustrate various embodiments of the present invention and are a part of the specification. The illustrated embodiments are merely examples of the present invention and do not limit the scope of the invention.

FIG. 1 is a diagram of a microstimulator and external controlling device according to principles described herein.

FIG. 2 is a diagram of a system for programming and controlling a microstimulator according to principles described herein.

FIG. 3 is an illustration of a user interface for a clinician programmer or computer used to program various components of the system illustrated in FIG. 2.

FIG. 4 is an illustration of a remote clinician programmer used to monitor a patient's experiments with an implanted stimulator.

FIG. 5 is a flowchart illustrating a method of enabling a patient to experiment with the parameters of an implanted stimulator according to principles described herein.

FIG. 6 is an illustration of a remote control or hand-held unit for controlling an implanted stimulator, where the unit includes a tutorial according to principles described herein.

FIG. 7 is a flowchart illustrating a method of using the remote control unit with tutorial features illustrated in FIG. 6.

Throughout the drawings, identical reference numbers designate similar, but not necessarily identical, elements.

### DETAILED DESCRIPTION

The present specification describes methods and systems that allow a patient to self-adjust the stimulation parameters for an electrical current being output by an implanted stimulator after an initial fitting session. In this way, the patient can help identify the stimulator settings that will be comfortable long-term and provide an optimal treatment for that patient's condition.

As used herein and in the appended claims, unless otherwise specifically denoted, the terms “stimulator” and “microstimulator” will be used interchangeably to refer to any implantable medical device that may be implanted within a patient and configured to transcutaneously communicate with an external device. As described above, an implanted stimulator delivers an electrical current to surrounding tissue to stimulate that tissue for therapeutic purposes.

The stimulating current that is output by an implanted stimulator is not constant, but is delivered in a regular cycle. Consequently, there are a number of parameters that characterize the current that is output by the implanted stimulator. For example, the stimulating current will have a frequency, amplitude and pulse width. These parameters can be adjusted to tailor the stimulation to the needs of a particular recipient patient. The stimulating current may also be delivered in bursts and have a duty cycle that describes the length and frequency of the current bursts.

Some patients receive a stimulator to control or mask chronic pain. In such patients, the stimulator may create a tingling sensation throughout a particular painful region of the body known as paresthesia. The size, intensity and character of the paresthesia may be controlled by adjusting the parameters of the stimulating current. For example, some patients may receive an implanted stimulator to treat relatively mild symptoms. In such a case the stimulating current may be weaker in amplitude, frequency and/or pulse width than in a patient who is being treated for stronger symptoms.

Consequently, when a patient first receives a new implanted stimulator, the operation of the stimulator must be configured and adjusted to suit that particular patient and his or her condition. This process is sometimes referred to as “fitting” the stimulator. A physician or other trained clinician will typically use both experience and intuition to provide initial parameters for the stimulating current of the implanted stimulator. Then, in consultation with the patient, these various parameters may be adjusted in an attempt to maximize the benefit of the stimulation for the patient while preventing the stimulator from causing any harm or discomfort to the patient.

This process, however, presents some difficulties. First, the fitting process may be very time consuming as efforts are made to find the optimal parameters for the stimulating current. A physician or other clinician may find it difficult to devote adequate time to the fitting process to reach a truly optimal result for the patient.

Additionally, the patient's level of comfort with the stimulation, or the effect on the patient of the stimulation, may change over an extended period. This may result, for example, from a change in the position of the implanted stimulator, its electrodes or catheters. Consequently, a stimulation setting that was effective or felt comfortable during a fitting session may not continue to be effective or may become uncomfortable in the days, weeks or months that follow the fitting session.

To address these issues, the present specification describes methods and systems that allow a patient to self-adjust the stimulation parameters of an implanted stimulator after an initial fitting session to help determine the optimal stimulation parameters for that patient and his or her condition. Typically, the patient will have a limited trial period in which to try different parameters settings. The patient will also typically have limits on the parameters that may be self-adjusted or the range within which parameters that may be self-adjusted to protect the patient from inadvertently causing tissue damage or damaging the implanted stimulator. In this way, a patient can help identify the stimulation parameters that will be comfortable long-term and provide an optimal treatment for that patient's condition.

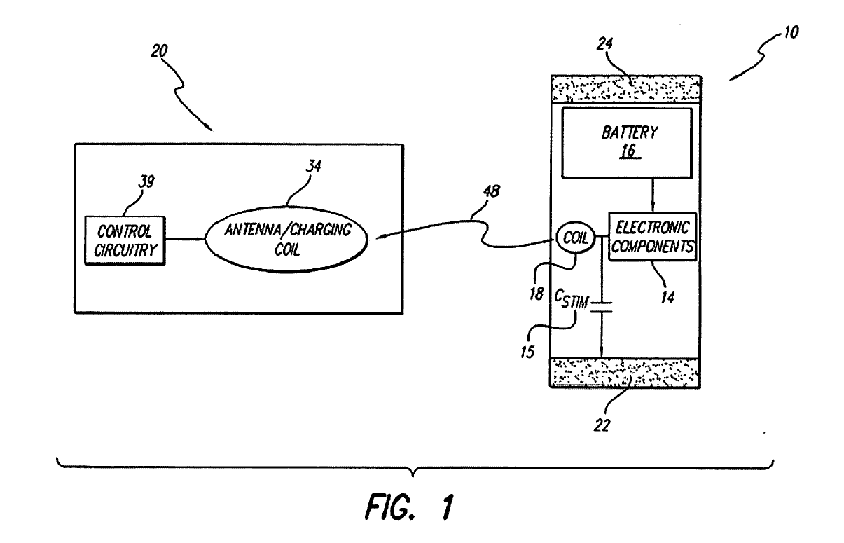

FIG. 1 shows an exemplary implantable stimulator (10) and an exemplary external device (20). As will be described in more detail below, the external device (20) may take any of several forms, including, but not limited to, a base station and chair pad or a remote control unit.

The implantable stimulator (10) may be any type of implantable medical device. For example, the implantable stimulator (10) may be an implantable microstimulator. Microstimulators are smaller than conventionally sized stimulators and are more easily implanted in a patient. Microstimulators may be injected through a large bore needle or placed via a small incision in the skin. An exemplary, but not exclusive, implantable microstimulator is the BION® microstimulator (Advanced Bionics® Corporation, Valencia, Calif.) which may be configured to stimulate tissue to alleviate urinary incontinence, reduce pain, or otherwise provide therapy for various disorders. Other examples of implantable stimulators include, but are not limited to, spinal cord stimulators (SCS), cochlear implants, and deep brain stimulators.

The implantable stimulator (10) is implanted in the target tissue area of a patient and the external device (20) may be used to communicate with the stimulator (10). Such communication may include, but is not limited to, transcutaneously transmitting data to the stimulator (10), receiving data from the stimulator (10), transferring power to the rechargeable battery (16) in the stimulator (10), and/or providing recovery power to the rechargeable battery (16) when the battery has been depleted to zero volts.

As illustrated in FIG. 1, the stimulator (10) may include a number of components including a rechargeable battery (16) configured to supply the stimulator (10) with power, a coil (18) configured to receive and/or emit a magnetic field that is used to communicate with the external device (20), a stimulating capacitor (15), and two or more electrodes (22, 24) configured to stimulate tissue with current. One or more of these components may be housed within a case (not shown).

The stimulator (10) may include additional and/or different electronic components (14) configured to perform a variety of functions as best serves a particular application. For example, in some embodiments, the stimulator (10) may include a memory unit in the electronic components (14). This memory unit can be used to store permitted ranges within which a patient may self-adjust the parameters governing the stimulating current output by the stimulator (10). As a safety measure, if the stimulator (10) is signaled to change a stimulation parameter beyond the permitted range for that particular parameter, the command can be ignored by the stimulator (10).

The exemplary external device (20) of FIG. 1 may include control circuitry (39) and an antenna/charging coil (34) configured to emit and/or receive a magnetic field that is used to communicate with the implantable stimulator (10). In one embodiment, the antenna/charging coil (34) and the stimulator's coil (18) communicate via a bidirectional telemetry link (48). The bidirectional telemetry link (48) may be known as a Radio Frequency (RF) telemetry link. The components of the external device (20) will be described in more detail below.

The external device (20) may be configured to perform any number of functions. For example, the external device (20) may be configured to transcutaneously charge the rechargeable battery (16) in the implanted stimulator (10). The external device (20) may also be configured to transcutaneously transmit data to the stimulator (10), receive data from the stimulator (10), and/or provide recovery power to the rechargeable battery (16) when the battery has been depleted to zero volts. The transmitted data may include configuration bits, programming bits, calibration bits, and/or other types of data. The signals that are sent between the external device (20) and the stimulator (10) may be modulated using frequency shift keying (FSK), on-off keying (OOK), or any other type of modulation scheme.

The functions performed by the external device (20) will vary as best serves the particular application of the stimulator (10) or the party using the external device (20). The shape and design of the external device (20) will likewise vary.

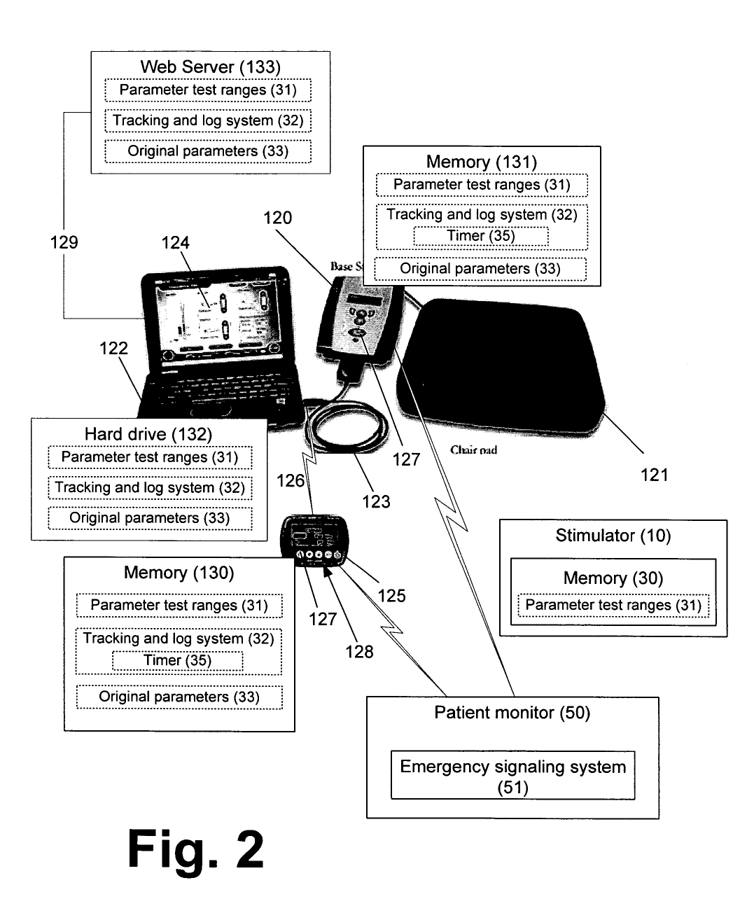

For example, as shown in FIG. 2, the external device (20) may be implemented as a chair pad (121) and a base station (120). The chair pad (121) is connected to, or in communication with, the base station (120) and includes the antenna/charging coil (34, FIG. 1) for communicating with the implanted stimulator. The base station (120) may include the control circuitry (39, FIG. 1) that controls the coil in the chair pad (121).

In use, the chair pad (121) may be placed on a chair and a patient who has an implanted stimulator (10, FIG. 1) may sit on the chair pad (121) to bring the implanted stimulator into proximity with the coil (34) in the chair pad (121). The chair pad (121) can then be used to recharge the battery (16, FIG. 1) in the stimulator and to transfer data between the base station (120) and the stimulator. Alternatively, the external device (20) may be housed within a casing that is worn by the patient near the surface of the skin. In general, the external device (20) may be any device configured to communicate with an implantable stimulator (10).

During a fitting session, the base station (120) may communicate with and be controlled by a computer or laptop (122). As shown in FIG. 2, a communications link (123) is provided between the computer (122) and the base station (120). This communications link (123) may be wired, as shown in FIG. 2, for example, or may be wireless. In some embodiments, the communications link (123) is an infrared data link. In a wireless infrared data link, the computer (122) may have a dongle comprising an infrared wireless transceiver that is plugged into a Universal Serial Bus (USB) port of the computer (122) and which communicates with a corresponding infrared wireless transceiver in the base station (120).

At the fitting session, the physician or other clinician will use an interface (124) on the computer (122) to adjust the parameters of the stimulation current being output by the implanted stimulator (10). These settings will be communicated to the stimulator (10) through the communications link (123), base station (120) and chair pad (121). The stimulator (10) will then function accordingly.

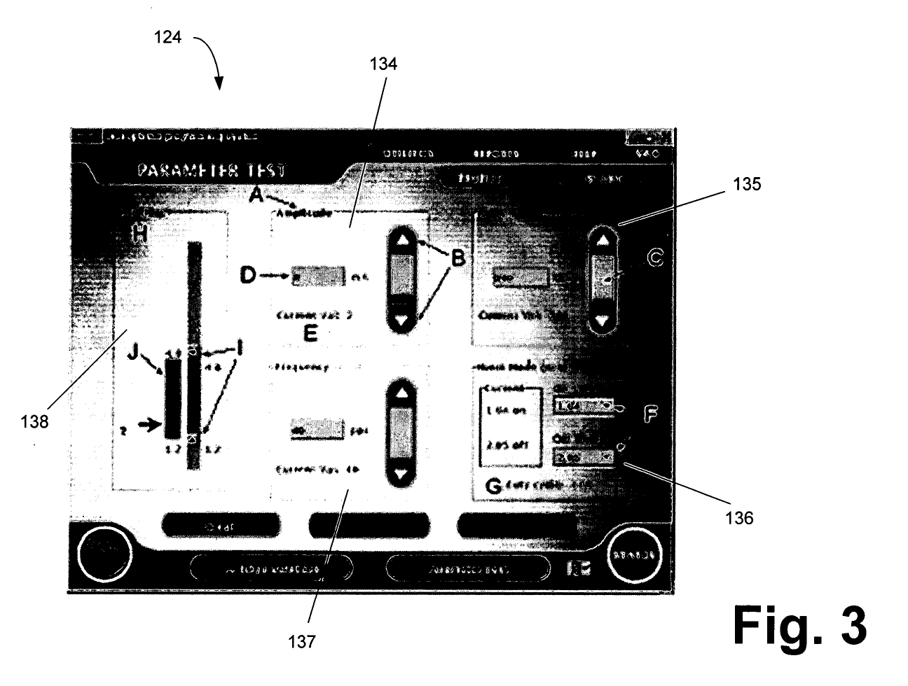

FIG. 3 is an exemplary illustration of one portion of the interface (124) used to control the implanted stimulator. For example, as shown in FIG. 3, the interface (124) includes amplitude controls (134), pulse width controls (135), frequency controls (137), etc., for controlling the stimulation parameters of the implanted stimulator.

The amplitude controls (134) are used for controlling the amplitude of a stimulation current being output by the implanted stimulator. These controls (134) may include an indication of the current amplitude setting, a field for entering a numeric amplitude value, for example, in milliamps and/or a scroll bar for changing the current amplitude setting.

As used herein and in the appended claims, a “scroll bar” is a bar that graphically shows a parameter setting relative to two ends or extremes. The scroll bar may be used to adjust the parameter by clicking or selecting, for example, with a mouse or other input device, a portion of the bar above or below the indicated parameter setting. This will correspondingly raise or lower the setting. The indicated parameter setting may also be dragged up or down within the scroll bar using the mouse or other input device. The scroll bar may also have up and down arrows associated therewith, for example at both ends of the scroll bar, that can be selected or clicked to adjust the indicated parameter setting in either direction.

The interface (124) also includes pulse width controls (135) for controlling the pulse width of a stimulation current being output by the implanted stimulator. Like the amplitude controls (134), these pulse width controls (135) may include an indication of the current pulse width setting, a field for entering a numeric pulse width value, for example, in microseconds (μs) and/or a scroll bar for changing the current pulse width setting.

The interface (124) also includes frequency controls (137) for controlling the frequency of a stimulation current being output by the implanted stimulator. Like the amplitude controls (134), these frequency controls (137) may include an indication of the current frequency setting, a field for entering a numeric frequency value, for example, in pulses per second (PPS) and/or a scroll bar for changing the current frequency setting.

In some examples, the interface (124) may also include controls for other characteristics or aspects of the stimulation current. For example, the interface (124) may include controls for controlling the shape of the pulses or waveform of the stimulation current. Controls may be included in the interface (124) for any characteristic or aspect of the stimulation current provided by the implanted stimulator.

The interface (124) may also include burst mode controls (136). These burst mode controls (136) may include, for example, an indication of the current burst mode settings, such as an amount of time on and an amount of time off that define the burst cycle. The burst mode controls (136) may also include fields or pull-down menus for adjusting both the time on and the time off of the burst cycle. A duty cycle value may also be calculated and displayed based on the time on/time off settings. Burst mode is most commonly used to test a new set of stimulation parameters for a patient.

The interface (124) of FIG. 3 may also include a set of maximum/minimum controls (138). These controls (138) are used to set maximum and/or minimum values beyond which the other parameters, e.g., amplitude, frequency, pulse width, burst length, may not be set. The maximum/minimum controls (138) may include one or more scroll bars that indicate the current setting of a parameter and the allowable range within which that parameter can be adjusted. The controls (138) can then be used to adjust the range within which the indicated parameter can be adjusted.

In some examples, the max/min controls (138) may include controls for setting a range of adjustment for each of the adjustable parameters. In other examples, one set of max/min controls may be provided and may be made active for a particular parameter by selecting corresponding controls for that parameter or by selecting the desired parameter from a list or menu.

Typically, the max/min controls (138) are available only to a physician or other trained clinician who has the expertise to establish a range of parameters within which a patient may adjust his or her stimulator. Consequently, access to the max/min controls (138) in the interface (124) may require entry of a password or other security token that demonstrates the user is qualified to adjust the max/min controls (138).

Returning to FIG. 2, during a fitting session, a physician or other clinician will place the patient having the stimulator (10) on the chair pad (121) or in proximity to some other external device (20, FIG. 1). This brings the stimulator (10) into sufficient proximity with the chair pad (121) that the base station (120) can communicate with the stimulator (10) through the electronics in the chair pad (121).

The clinician, using the computer (122) with the interface (124), then establishes an original set of parameters for the stimulating current output by the stimulator (10). This may be done using experience and intuition initially, and then adjusting the parameters in consultation with the patient as to what settings are comfortable and how various parameter adjustments improve or decrease the apparent value of the treatment with the stimulator (10). Within the time allowed for the initial fitting session, the clinician will establish a set of stimulation parameters that seem to provide the best overall effect for the patient.

These “original parameters” (33) may be stored in any or all of several locations. For example, the original parameters (33) established during the fitting session will be stored on the hard drive (132) in the computer (122) used by a clinician during the fitting session. The original parameters (33) may also be stored on a web server (133) on the Internet, where the computer (122) has a connection (129) to the Internet or World Wide Web. Use of the web server (133) will be described in further detail below. The original parameters (33) may also be stored in the memory (131) of the base station (120) and/or in the memory (130) of a remote control unit (125).

The remote control unit (125) is an external device (20, FIG. 1) that can be provided to a patient to control an implanted stimulator (10) during or after the initial fitting session. Consequently, the remote control unit (125) includes a coil which with to communicate transcutaneously with the implanted stimulator (10) in the manner described above. The remote control unit (125) may be used to transmit data to or receive data from an implanted stimulator (10) and can be used, in some examples, to recharge an implanted stimulator (10). Thus, the remote control unit (125) can be used in concert with, or in place of, the base station (120) and chair pad (121).

The remote control unit (125) will also include an interface (126) for communicating with the computer (122). This interface (126) may be, for example, a wired or wireless connection. The interface (126) may make use of the same infrared dongle used by the computer (122) to communicate with the base station (120) in some examples. The interface (126) may also be a Universal Serial Bus (USB) or other wired interface.

Thus, during the fitting session, the remote control unit (125) will be programmed and prepared for subsequent use by the patient in controlling and adjusting the implanted stimulator (10). This may include storing the original parameters (33) established during the fitting session in the memory (130) of the remote control unit (125).

The remote control unit (125) includes a user interface (128) with which a patient can control the remote control unit (125) to send commands to, or retrieve data from, the implanted stimulator (10). This user interface (128) of the remote control unit (125) may include any device or devices for allowing a patient to send commands to, retrieve data from, send power to or otherwise operate the implanted stimulator (10). Such devices may include, but are not limited to, buttons, a keypad, a joystick, dials, knobs, switches, sliders, a display and/or a touch-sensitive display device such as a liquid crystal touch-sensitive display.

This user interface (125) for the remote control unit (125) will, in some examples, include a reset button or other reset control (127). This reset control (127), when actuated, will retrieve the original parameters (33) from memory (130) and will signal the stimulator (10) to return to those original parameters (33). In this way, a patient who has been experimenting with the stimulation parameters of his or her stimulator (10) can always return to the original settings or parameters established at the initial fitting session for the stimulator (10).

Similarly, the base station (120) may also include a user interface with a reset control (127). As with the remote control unit (125), this reset control (127), when actuated, will retrieve the original parameters (33) from memory (131) and will signal the stimulator (10) to return to those original parameters (33) established at the initial fitting session for the stimulator (10).

As part of the initial fitting session, the physician or clinician fitting the stimulator (10) will decide whether it would be advantageous and safe to allow the patient to adjust the stimulation parameters of the stimulator (10), e.g., amplitude, pulse width, frequency, burst pattern, pulse shape, etc. As indicated above, the original parameters (33) established at the fitting session can often be improved upon as the patient reaction to the stimulator (10) changes over time.

If the clinician decides to allow patient self-adjustment of the stimulation parameters. The clinician will access the max/min controls (130, FIG. 3) of the interface (124). As indicated above, this may require a password or other security token. The clinician then uses the max/min controls (138) to establish which parameters a patient may self-adjust and the permissible range or ranges within which that parameter or parameter may be adjusted by the patient.

These parameter test ranges (31) are then stored on the hard drive (132) of the computer (122). The parameter test ranges (31) are also transmitted to and stored in any of several other places. For example, the parameter test ranges (31) may be stored on the web server (133). The parameter test ranges (31) may also be stored in the memory (131) of the base station (120) and the memory (130) of the remote control unit (125).

The parameter test ranges (31) may also be stored in a memory (30) in the implanted stimulator (10) itself. Either the base station (120) or the remote control unit (125) may be used to transmit and store the parameter test ranges (31) in the stimulator (10). If the parameter test ranges (31) are stored in the stimulator (10) itself, the stimulator (10) may also be programmed to ignore any command to adjust the stimulation parameters outside of the permissible test ranges (31).

The clinician may also determine a set period of time during which the patient will be allowed to adjust the stimulation parameters of the stimulator (10). This period of time may be days, weeks, months, etc. If a time limit is set, it is tracked by a timer (35) in either the base station (120) or the remote control unit (125).

When the timer (35) indicates that the time for patient self-adjustment has expired, the clinician can program the system to do any of several things. For example, the remote control unit (125) or the base station (120) may signal the stimulator (10) to return to the original parameters (33) of the fitting session when the timer (35) has expired. Alternatively, the current stimulation parameters being used when the timer (35) expires may remain in effect. In another alternative, the stimulator (10) may be deactivated when the timer (35) has expired. In any event, the patient may or may not be required to again visit the clinician for further evaluation and decisions about continued treatment when the timer (35) has expired.

Following the initial fitting session, as described herein, the patient will be allowed to adjust the stimulation parameters of the stimulator (10) in an attempt to help identify the parameter set that will be the most effective and comfortable for the patient over time. After the patient leaves the initial fitting session, the patient may have access to some or all of the system shown in FIG. 2. For example, the patient may have only the remote control unit (125) with which to control the stimulation parameters of the stimulator (10). Alternatively or additionally, the patient may have a base station (120) and chair pad (121) with which to control and recharge the stimulator (10) after the initial fitting session.

In some embodiments, the patient may even have a computer (122) with which the remote control unit (125) and/or base station (120) can communicate. This computer may or may not have the entire interface (124) described above as installed on the clinician programmer (122) that was used for the initial fitting session. If present, the max/min controls and other controls of the interface (124) that should only be operated by a clinician will be locked out or disabled.

It is useful for a patient to have a computer (122) communicating with the remote control unit (125) and/or base station (120) so that data on the self-adjustment of the stimulator (10) can be uploaded to the web server (133) over an Internet connection (129). As will be described in more detail below, the web server (133) can then be accessed by the patient's physician or clinician to remotely supervise and/or control the patient's experiments with the stimulator (10).

Various components of the system shown in FIG. 2 may also include a tracking and log system (32). For example, the tracking and log system (32) may be included in the remote control unit (125), base station (120) and/or computer (122). This tracking and log system (32) is used to track the changes to stimulation parameters input and tried by the patient. In some examples, all the parameter adjustments input by the patient will be automatically tracked and recorded by the tracking and log system (32). This may be the full extent of the operation of the tracking and log system (32).

In other examples, the tracking and log system (32) may allow the patient to input comments associated with each particular set of stimulation parameters that describe how those parameters subjectively feel to the patient, e.g., the effectiveness and comfort of those particular stimulator settings. If the patient is using a remote control unit (125), the remote control unit interface (128) may allow the patient to input comments to the tracking and log system (32). For example, the remote control unit (125) may include an alphanumeric keypad or keyboard or a touch sensitive display similar to a Personal Digital Assistant. In any event, the remote control unit (125) may have the capacity to receive patient comments and store the same in the tracking and log system (32). Similarly, the base station (120) may incorporate a user interface that allows the patient to input comments to a tracking and log system (32).

Additionally, if the patient's system includes a computer (122), the computer (122) may be used to enter comments regarding particular parameter sets to a tracking and log system (32). If the patient is using a remote control unit (125) or a base station (120), comments in the tracking and log system of the remote control unit (125) or the base station (120) may be transferred (123, 126) to the computer (122) for back-up and long-term storage.

Additionally, the comments and other data in the tracking and log system (32) may be uploaded to a corresponding tracking and log system (32) on the web server (133). For example, if the comments and record of parameter sets is stored on or transferred to the computer (122), the computer (122) can upload the data to the web server (133) over the Internet connection (129). Alternatively, either the remote control unit (125) or the base station (120) may transmit data to the web server (133) without using the computer (122).

The system of FIG. 2 may also include a patient monitor system (50). This patient monitor may include any device for monitoring a vital sign or other condition of the patient. For example, the patient monitor (50) may include a heart rate monitor, a blood pressure monitor, a blood oximeter, etc., for monitoring the condition of a patient during self-adjustment of the stimulation parameters of the implanted stimulator (10).

The patient monitor (50) may transmit data to the tracking and log system (32) in the other elements of the system, such as the remote control unit (125) or base station (120). In this way, the data regarding the patient's condition from the monitor (50) can be included with corresponding stimulation parameter sets in the tracking and log system (32).

The patient monitor (50) may also include an emergency signaling system (51). If the monitored vital signs of the patient exceed established safety thresholds stored in the monitor (50), the emergency signaling system (51) may automatically signal a medical emergency and call for help. For example, the emergency signaling system (51) may call a “911” operator using a wireless phone unit or standard phone line. In other examples, the emergency signaling system (51) may signal a residential alarm or monitoring service. In any event, the emergency signaling system (51) includes some means for signaling an emergency and calling for help in the event that patient self-adjustment of stimulation parameters for the implanted stimulator (10) results in one or more monitored patient vital signs exceeding prescribed limits.

The emergency signaling system (51) may also signal for the deactivation of the stimulator (10). For example, the emergency signaling system (51), upon detecting a patient vital sign that exceeds a safe range, can signal the remote control unit (125) or the base station (120) to deactivate the stimulator (10).

In some embodiments, the emergency signaling system (51) may signal the remote control unit (125) or base station (120) to return the stimulator (10) to the original stimulation parameters (33). This may occur, for example, if monitored patient vital signs indicate stress to the patient which is not yet at a dangerous or emergency level. In some examples, the patient monitor (50) may control the stimulator (10) directly.

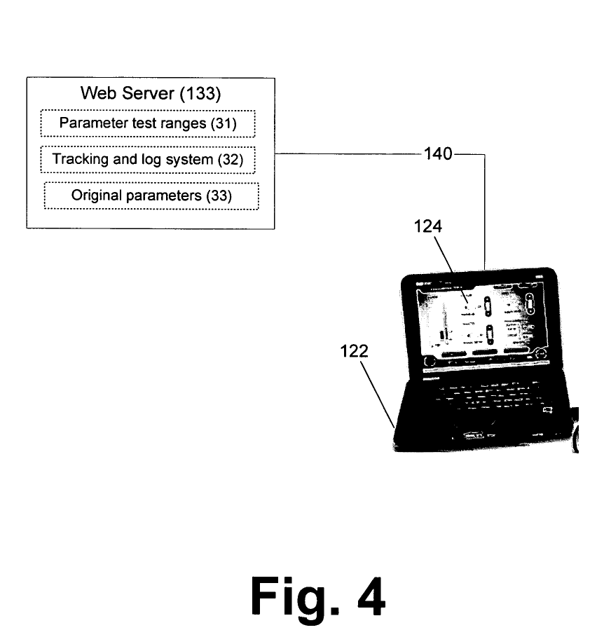

FIG. 4 is an illustration of a remote clinician programmer used to monitor a patient's experiments with an implanted stimulator. As shown in FIG. 4, a computer (122) used by a physician or other clinician treating the patient with the implanted stimulator can be connected to the Internet or World Wide Web, via an Internet connection (140). The computer (122) may be a general purpose computer or a dedicated clinician programmer for working with implanted stimulators.

With the computer (122), the clinician can access the web server (133). As explained above, in some embodiments, the history of stimulation parameters tried by the patient during his or her experimentation with the stimulator, any appended comments and, in some examples, a corresponding record of the patient's vital signs as reported by the patient monitor (50, FIG. 2) will be uploaded to the web server (133) by the patient's system. Consequently, during or after the trial period in which the implant patient is allowed to self-adjust the stimulation parameters as described above, the clinician overseeing the patient's treatment can access the tracking and log system (32) at the web server (133) and obtain all the records generated by the patient's experimentation with the implanted stimulator.

With this information, the clinician may make any desired adjustments to the treatment. For example, the clinician may extend or decrease the length of the trial period during which the patient is allowed to self-adjust the stimulation parameters. The clinician may change the original parameters (33) or change the parameter test ranges (31). Any changes made to the treatment program on the web server (133) will then be communicated to and implemented in the patient's system, e.g., computer (122, FIG. 2); remote control unit (125, FIG. 2) or base station (120, FIG. 2).

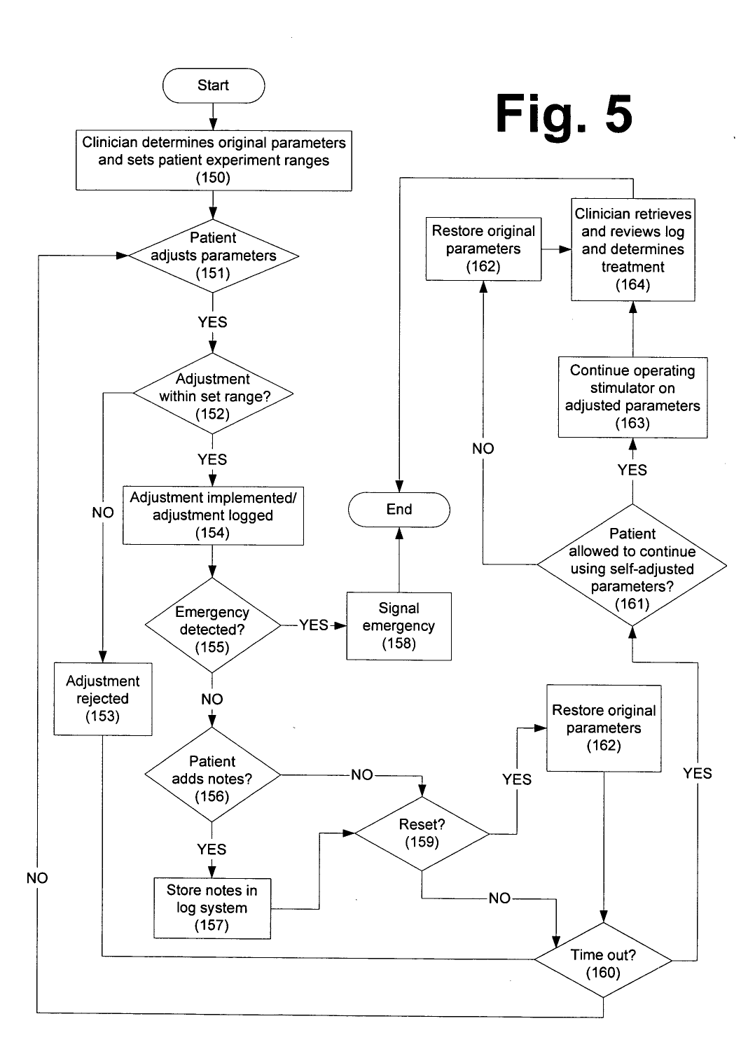

FIG. 5 is a flowchart illustrating a method of enabling a patient to experiment with the parameters of an implanted stimulator according to principles described herein. As shown in FIG. 5, the method begins when a clinician has a fitting session with the implant patient and establishes the initial stimulation parameters for the implanted simulator and the ranges within which the patient can make experimental adjustments (step 150).

After the patient leaves the fitting session, the patient can adjust the stimulation parameters within the ranges set by the clinician as described above. This allows the patient an extended period in which to try out and adjust the stimulation parameters to identify those parameters that will be effective and comfortable long-term. The result for the patient may then be significantly better than could be achieved in the limited duration of the initial fitting session with the clinician.

If the patient enters an adjustment to the stimulation parameters (determination 151), the adjustment is checked to see if it is within the permissible range set by the clinician (determination 152). If the adjustment is outside the permissible range (determination 142), the adjustment is rejected and not implemented by the system (step 153). If, however, the adjustment is within the range allowed for patient experimentation (determination 152), the adjustment is implemented by the system so that the stimulator implements or begins operating based on the new stimulation parameters (step 154). Additionally, the adjustment is recorded and logged by the system to keep a record of the patient's experimentation with the stimulator (step 154).

As the patient is making adjustments to the stimulation parameters, an emergency could potentially occur. As described above, a patient monitor (50, FIG. 2) may be used to determine whether the patient is experiencing an emergency. If an emergency is detected (determination 155), an emergency signal may be issued as described above (step 158) and the stimulator is deactivated or returned to the original stimulation parameters.

As the patient makes adjustments and those adjustments are logged (step 154), the patient may also input notes or comments (step 156) on the various parameter sets tried. The patient may comment, for example, on the effectiveness, comfort and character of the stimulation or paresthesia resulting from a particular set of stimulation parameters. Any such notes will be stored in the log and tracking system (157) along with the record of different stimulation parameter sets tried by the patient.

During this experimentation with the stimulator, the patient will have the option of resetting the stimulator to the stimulation parameters originally set by the clinician in the initial fitting session. Various reset mechanisms are described above in connection with the remote control unit or base unit the patient may be using to control the stimulator. If a reset is entered (determination 159), the original parameters are restored (step 162), i.e., a command to return to the original parameters is transmitted to, and implemented by, the implanted stimulator.

Typically, the clinician will also set a timer with a specified time period in which the patient will be allowed to adjust the stimulation parameters. This is not necessarily so, the patient may be allowed to adjust the stimulation parameters indefinitely or permanently. However, if a timer is set, eventually the time for self-adjustment of stimulation parameters will end. If the timer for stimulator adjustment times out (determination 160), the system may stop taking and implementing any parameter adjustments.

When the timeout occurs (determination 160), the patient may (determination 161) be allowed to continue using the stimulator with the last input parameter set (step 163) or the stimulator may revert to the original parameters (step 162). In either event, the clinician will then retrieve the log and tracking data of the patient's experimentation.

As described above, the clinician may access the log and tracking data by accessing the web server (133, FIG. 2). Alternatively, the clinician may have the patient return the remote control unit (125, FIG. 2) or the base station (120, FIG. 2) and may download the log and tracking data directly from the remote control unit (125, FIG. 2) or base station (120, FIG. 2). Upon reviewing that data, the clinician can decide on the future course of treatment for the patient (step 164).

For example, the clinician may then decide to allow the patient to continue self-adjusting the stimulator. The clinician may, alternatively, be able to confidently set permanent stimulation parameters for the patient. Either course of action may be done in an office visit or remotely via the web server (133, FIG. 2). By accessing the web server, the clinician can obtain the record of the patient's experimentation with the stimulator and/or send commands to the stimulator as described above to, for example, extend the trial period, limit the ranges in which the parameters can be adjusted, set permanent stimulation parameters, etc.

As an alternative to the web server (133, FIG. 2), the clinician may have other methods of remotely communicating with the patient's system and issuing commands or adjusting controls. For example, the clinician may contact some element of the patient's system (e.g., computer 122, remote control 125, base station 120) using a telephone line, wireless telephone link, etc. Any data link may be used for the clinician to communicate with the patient's system.

As will be understood by those skilled in the art the systems and methods described above may be applied to a combination of two or more stimulators that have been implanted in a patient. Thus, a patient may be able to experiment with the settings for multiple implanted stimulators. The systems and methods described can also be applied to external, as opposed to internal implanted, stimulators.

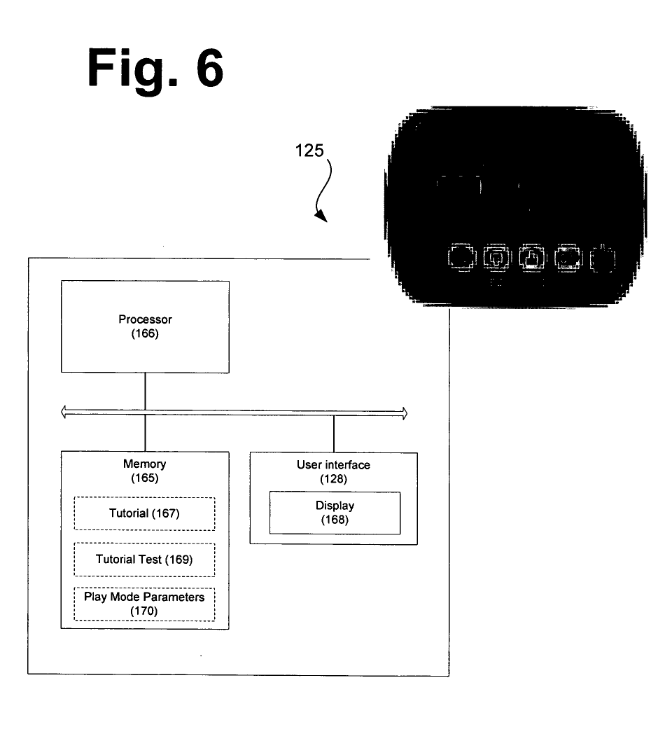

FIG. 6 is an illustration of a remote control or hand-held unit for controlling an implanted stimulator, where the unit includes a tutorial according to principles described herein. The advantages of allowing a patient to experiment with the settings of an implanted stimulator after the initial fitting session have been well described above. However, some patients may require some training and education before being able to safely and effectively experiment with stimulation parameters. Some patients may simply need basic training to be able to correctly operate an external device (20, FIG. 2) such as the remote control unit described above.

Consequently, as shown in FIG. 6, an external device, such as a remote control unit (125) may include a tutorial (167) stored in memory (165). When this tutorial is invoked, information is displayed on the display device (168) of the user interface (128) to teach the patient about the remote control unit (125) and its operation. For example, various screens of the tutorial may give general information about the remote control unit (125), its features and general use tips such as what to do if the remote control unit is lost, what to do when the batteries run low, etc.

The tutorial may also include the same interface (124, FIG. 2) used to adjust stimulation parameters. With the tutorial executing, the patient can see how adjustments to stimulation parameters are made and can experiment with the interface (124, FIG. 2) without actually adjusting stimulation parameters.

After the tutorial has run, the remote control unit (125) may administer a tutorial test (169), also stored in memory (165). The tutorial test (169) will provide questions that are posed and answered by the patient through the user interface (128). If the patient fails the test, the remote control unit (125) may replay the tutorial (167) or specific portions of the tutorial based on what portions of the test (169) the patient failed to answer properly.

When the patient satisfactorily passes the tutorial test (169), the remote control unit (125) may enter a “play” mode. In this mode, the patient will be able to actually adjust stimulation parameters in the same manner as described above. However, the limits (170) within which the stimulation parameters are adjusted in the “play” mode will be very limited.

Portions of the tutorial (167) will also be active during the “play” mode to provide the patient with information about the adjustments to the stimulation parameters. For example, as the patient adjusts stimulation parameters in the “play” mode, the tutorial (167) may instruct the patient as to how various adjustments affect battery life and the recharging requirements of the stimulator. This will allow the patient to make informed decisions about the tradeoffs between pain relief or stimulation and convenience.

This feature of the tutorial may continue to operate, for example, during the trial period in which the patient is actually experimenting over an extended period with adjustments to the stimulation parameters. As the user is making actual adjustments to the stimulation parameters, it may be helpful for a battery-drain model on the remote control unit to advise the patient how the adjustments will affect battery life and the frequency of recharging cycles. Again, this will allow the patient to make informed decisions about the tradeoffs between pain relief or stimulation and convenience.

The tutorial (167) may also advise the patient of which parameters are likely to affect the intensity, breadth or character of the resulting stimulation or paresthesia. These portions of the tutorial (167) may appear as pop-ups on the display (168) as parameters are adjusted.

The patient may work in the “play” mode prior to, or during, an initial fitting session. After experiencing the “play” mode, the patient will be well prepared for the trial period described above during which the patient experiments with the stimulation parameters over, perhaps, a wide range and subsequent to the initial fitting session.

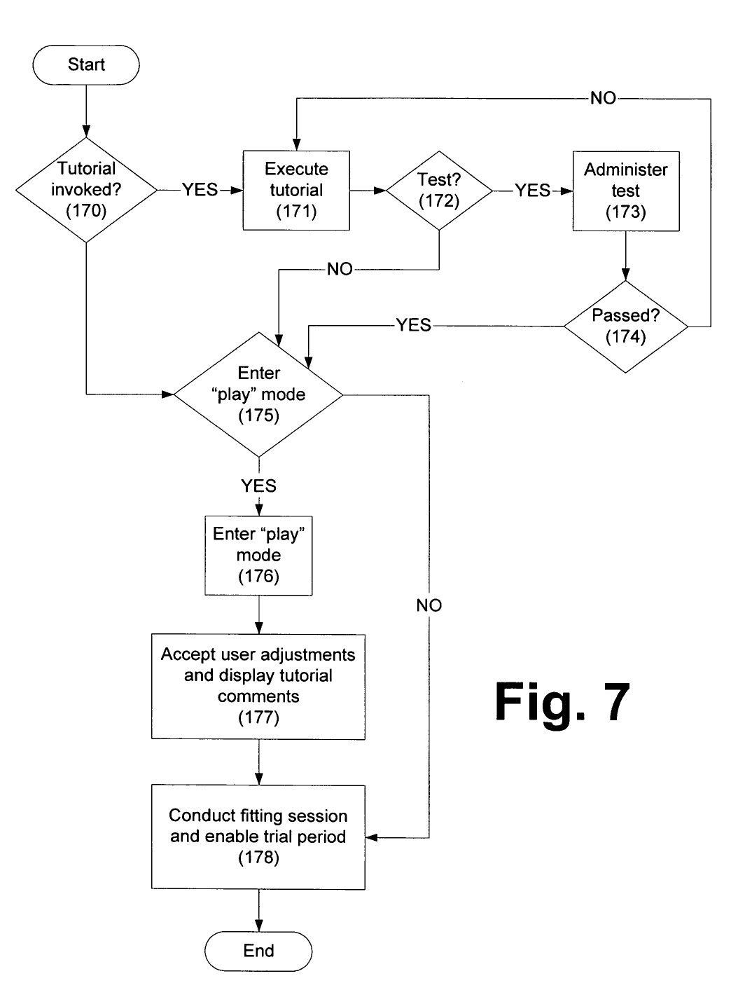

FIG. 7 is a flowchart illustrating a method of using the remote control unit with tutorial features illustrated in FIG. 6. As shown in FIG. 7, when the patient receives the remote control unit, the tutorial may be invoked (determination 170). However, this is not necessarily so. Some patients may already be sufficient familiar with the technology so as not to need or want to experience the tutorial. If the tutorial is not invoked (determination 170), the remote control unit may go directly into the “play” mode (determination 175) described above.

If the tutorial is invoked (determination 170), the tutorial is executed (step 171) and provides the user with the training and instruction regarding the remote control unit and implanted stimulator as described above. When the tutorial is completed, a test may be administered (determination 172) to assess the patient's understanding and comfort with the remote control unit. In some cases, the test may be skipped.

If the test is administered (determination 172), the patient sees and responds to the questions of the test (step 173). Depending on some predetermined standard, the patient will be judged to have passed or failed the test (determination 174). A pass may require a certain number of correct responses or that all responses are correct. If the patient fails the test (determination 174), the tutorial or potions of the tutorial can be replayed (step 171). The test may then be re-administered until the patient has learned enough about the remote control unit to advance to the “play” mode or to the fitting session.

Eventually, the remote control unit may enter the “play” mode (determination 175). However, the “play” mode is not required. The patient may choose to skip the “play” mode, or the clinician may not have the patient experience the “play” mode.

As described above, the “play” mode (step 176) allows the patient to make actual adjustments to stimulation parameters (step 177). The “play” mode also includes portions of the tutorial as described above the supplement the patient's experimentation with the stimulation parameters.

Finally, the patient will have the fitting session (step 178). At the fitting session, a trial period may be enabled, as described above, that allows the patient to experiment with stimulation parameters over and extended period of time. The method of, for example, FIG. 5 may then be implemented.

In some embodiments, the tutorial (167) and other features of the remote control unit (125) can be accessed and used by the patient at any time, and not just prior to or in conjunction with the initial fitting session. This allows the patient to refresh his or her understanding of the remote control unit and its features whenever desired.

The preceding description has been presented only to illustrate and describe embodiments of the invention. It is not intended to be exhaustive or to limit the invention to any precise form disclosed. Many modifications and variations are possible in light of the above teaching. It is intended that the scope of the invention be defined by the following claims.

1. A method of adjusting operation of a stimulator for treatment of a patient using said stimulator, said method comprising:
adjusting stimulation parameters of said stimulator subsequent to a fitting session with a clinician,
wherein said adjusting is performed by said patient who has said stimulator.

2. The method of claim 1, wherein said stimulation parameters comprise amplitude, frequency or pulse width.

3. The method of claim 2, wherein said stimulation parameters comprise pulse shape or burst duty cycle.

4. The method of claim 1, further comprising limiting which stimulation parameters are available for adjustment by said patient.

5. The method of claim 1, further comprising setting limits within which one or more of said stimulation parameters can be adjusted by said patient.

6. The method of claim 1, further comprising storing a log of adjustments to said stimulation parameters made by said patient.

7. The method of claim 6, further comprising recording patient comments in said log.

8. The method of claim 6, further comprising uploading said log to a web server.

9. The method of claim 6, further comprising downloading said log for access by a clinician treating said patient.

10. The method of claim 1, further comprising resetting said stimulator to original stimulation parameters established by said clinician at said fitting session.

11. The method of claim 1, further comprising monitoring a patient's condition during said adjustment.

12. The method of claim 11, further comprising storing a log of said patient's condition being monitored.

13. The method of claim 12, further comprising transmitting said log to said clinician treating said patient.

14. The method of claim 11, further comprising signaling an emergency if said patient condition being monitored exceeds a predetermined threshold.

15. The method of claim 14, further comprising deactivating said stimulator in response to said emergency.

16. The method of claim 14, further comprising returning said stimulator to initial stimulation parameters established during said clinician at said fitting session in response to said emergency.

17. The method of claim 1, further comprising timing a trial period during which said patient adjustment of said stimulation parameters is permitted.

18. The method of claim 17, further comprising, when said trial period has elapsed, returning said stimulator to initial stimulation parameters established by said clinician at said fitting session.

19. The method of claim 17, further comprising, when said trial period has elapsed, deactivating said stimulator.

20. The method of claim 17, further comprising, when said trial period has elapsed, accepting no further adjustments of said stimulation parameters.

21. The method of claim 1, wherein said stimulator is an implanted stimulator and said adjusting stimulation parameters includes transcutaneously communicating with said implanted stimulator.

22. The method of claim 1, wherein said adjusting is performed by said patient with a remote control unit.

23. The method of claim 22, further comprising providing a tutorial with a user interface of said remote control unit to instruct said patient about said stimulation parameters.

24. The method of claim 23, further comprising testing said patient with said remote control unit following said tutorial to assess patient understanding of subjects covered by said tutorial.

25. A system for patient adjustment of operation of a stimulator used by the patient subsequent to a fitting session with a clinician, said system comprising:
an external device for communicating with said stimulator; and
a user interface of said external device configured to receive input from said patient to adjust stimulation parameters of said stimulator.

26. The system of claim 25, wherein said external device comprises a remote control unit.

27. The system of claim 25, wherein said external device comprises a base station and chair pad.

28. The system of claim 25, wherein said stimulation parameters comprise amplitude, frequency or pulse width.

29. The system of claim 25, wherein said stimulation parameters comprise pulse shape or burst duty cycle.

30. The system of claim 25, further comprising limits in said external device on which stimulation parameters are available for adjustment by said patient.

31. The system of claim 25, further comprising limits in said external device within which one or more of said stimulation parameters can be adjusted by said patient.

32. The system of claim 25, further comprising a memory unit in said external device for storing a log of adjustments to said stimulation parameters made by said patient.

33. The system of claim 32, wherein said user interface receives, and said memory unit stores, patient comments for said log.

34. The system of claim 32, further comprising a communications link for transmitting said log for access by said clinician treating said patient.

35. The system of claim 34, wherein said communications link is an Internet connection for transmitting said log to a web server.

36. The system of claim 25, wherein said user interface further comprises a reset control for resetting said stimulator to original stimulation parameters established by said clinician at said fitting session.

37. The system of claim 25, further comprising a patient monitor for monitoring a patient's condition during said adjustment.

38. The system of claim 37, further comprising a memory for storing a log of said patient's condition being monitored.

39. The system of claim 38, further comprising a communications link for transmitting said log to said clinician treating said patient.

40. The system of claim 37, further comprising an emergency signaling system for signaling an emergency if said patient condition being monitored exceeds a predetermined threshold.

41. The system of claim 25, further comprising a timer in said external device for timing a trial period during which said patient adjustment of said stimulation parameters is permitted.

42. The system of claim 25, wherein said stimulator is an implanted stimulator and said external device communicates with said implanted stimulator adjusting stimulation transcutaneously.

43. The system of claim 25, further comprising a tutorial stored electronically in said external device for instructing said patient through said user interface about said stimulation parameters or said external device.

44. A remote control unit for patient control of a stimulator used by said patient, said remote control unit comprising:
a processor;
a user interface; and
a memory unit comprising a tutorial for instructing said patient through said user interface about said remote control unit or said stimulator.

45. The unit of claim 44, further comprising a test stored in said memory unit for testing said patient through said user interface to assess said patient's understanding of subject matter covered by said tutorial.

46. The unit of claim 45, wherein a “play” mode is enabled if said patient passes said test, said “play” mode allowing said patient to make adjustments to actual stimulation parameters of said stimulator.

47. A system for allowing a patient who is being treated with a stimulator to adjust operation of said stimulator subsequent to a fitting session with a clinician, said system comprising:
means for patient-adjustment of stimulation parameters of said stimulator subsequent to said fitting session with a clinician; and
means for recording adjustments to said stimulation parameters made by said patient subsequent to said fitting session.

48. The system of claim 47, wherein said stimulation parameters comprise amplitude, frequency or pulse width.

49. The system of claim 47, wherein said stimulation parameters comprise pulse shape or burst duty cycle.

50. The system of claim 47, further comprising means for limiting which stimulation parameters are available for adjustment by said patient.

51. The system of claim 47, further comprising means for limiting a range within which one or more of said stimulation parameters can be adjusted by said patient.

52. The system of claim 47, further comprising means for receiving and recording patient comments regarding said patient-adjustment of said stimulation parameters.

53. The system of claim 47, further comprising means for resetting said stimulator to original stimulation parameters established by said clinician at said fitting session.

54. The system of claim 47, further comprising means for monitoring a patient's condition during said adjustment.

55. The system of claim 47, further comprising means for signaling an emergency if said patient condition being monitored exceeds a predetermined threshold.

56. The system of claim 47, further comprising means for timing a trial period during which said patient adjustment of said stimulation parameters is permitted.

57. The system of claim 47, wherein said stimulator is an implanted stimulator and said adjusting stimulation parameters includes transcutaneously communicating with said implanted stimulator.
